(19) United States
(12) Patent Application Publication
(10) Pub. No.: US 2011/0287051 A1
(43) Pub. Date: Nov. 24, 2011

(54) PARAPOXVIRUS VECTORS

(75) Inventors: Olivier Michel Martinon, Richland, MI (US); Nanda Kumar D Reddy, Holly, MI (US)

(73) Assignee: PFIZER INC., New York, NY (US)

(21) Appl. No.: 13/111,020

(22) Filed: May 19, 2011

Related U.S. Application Data
(60) Provisional application No. 61/346,988, filed on May 21, 2010.
(60) Provisional application No. 61/414,287, filed on Nov. 16, 2010.

(51) Int. Cl. A61K 39/275; C12Q 1/70; C12N 7/01; C12N 15/63; A61P 31/20; A61P 37/04

(52) U.S. Cl. 424/199.1; 435/235.1; 435/471; 435/5

(57) ABSTRACT

The present invention relates to recombinant parapoxviruses which carry in their genomes comprising heterologous DNA derived from a rabies virus, to the preparation of such constructs, and to their use in immunogenic compositions and vaccines. It further relates to the use of recombinant parapoxviruses for diagnostics.

### CROSS-REFERENCE TO RELATED APPLICATIONS

This application claims priority, under 35 U.S.C. §119(e), to U.S. Provisional Application Ser. Nos. 61/346,988 and 61/414,287, filed on May 21, 2010 and Nov. 16, 2010, respectively, the entire disclosures of which are incorporated herein by reference.

### FIELD OF THE INVENTION

The present invention relates to recombinant parapoxviruses that contain heterologous DNA derived from a rabies virus (RV) and to their use in immunogenic compositions and vaccines. It also relates to methods for vaccinating against, treating, or preventing disease caused by rabies virus. It further relates to the use of recombinant parapoxviruses for diagnostics.

### BACKGROUND OF THE INVENTION

Viruses of the Poxyiridae family are oval, quite large, double-stranded DNA viruses. The genus Parapoxvirus (PPV) is included among these viruses. They measure about 220-300 nm long by 140-170 nm wide. They possess a unique spiral coat that distinguishes them from the other poxviruses.

The PPV are divided into three different species. However, it has still not been clarified whether these viruses are autonomous species within the parapoxvirus genus or whether they are the same species. The first species, Parapoxvirus ovis, is regarded as the prototype of the genus. It is also called ecthyma contagiosum virus, contagious pustular dermatitis virus, or orf virus. The second, Parapoxvirus bovis 1, is also called bovine papular stomatitis virus or stomatitis papulosa virus. The third, Parapoxvirus bovis 2, is also called udderpoxvirus, paravaccinia virus, pseudocowpox virus, or milker's nodule virus.

Parapoxvirus species are endemic in ruminants. PPVs have been found in red deer, reindeer, red squirrels, and harbor seals. Infections with PPV can cause local diseases in both animals and man. The zoonotic hosts of PPV species are sheep, goats, and cattle. They cause infections in humans through direct contact with infected animals, reacting with localized epidermal lesions which heal without scaring. Prophylactic measures, such as vaccines, can be used to control the diseases.

Vectors for expressing foreign genetic information based an avipox, racoonpox, capripox, swinepox, or vaccinia virus have been described previously (see U.S. Pat. No. 5,942,235 and U.S. Pat. No. 7,094,412). Parapoxviruses represent different candidates that can be used in vector vaccines. However, because of morphological, structural, and genetic differences between the individual genera of the poxviruses, the methods used for these pox viruses cannot be used for Parapoxvirus. An example of such differences is that ORFV is missing a thymidine kinase (TK) gene, which is used for selection of recombinants in the different orthopoxviruses. Also, some poxviruses have the ability to agglutinate erythrocytes, which is mediated by way of a surface protein, the haemagglutinin, while Parapoxviruses do not.

PPV can have an immunomodulatory effect because they stimulate generalized (non-specific) immune reactions in vertebrates. They have been used successfully in veterinary medicine for increasing general resistance in animals. They can be combined with a homologous and/or heterologous antigen to provide vaccines that have a pathogen-specific effect which lasts for months to years, as well as a rapid non-pathogen-specific effect.

Parapoxvirus ovis has been used previously as a vector, as described in U.S. Pat. No. 6,365,393 and Rziha et al., 2000, J. Biotechnol, 83, 137-145. It offers remarkable advantages when used as a vector, including a very narrow host range, lack of systemic infection, short-term vector-specific immunity (allowing repeated immunizations), early vaccination (induction of immunity can be started in presence of maternal antibodies), and beneficial immune modulating properties. The present invention relates to using Parapoxvirus as a vector for heterologous DNA derived from rabies virus.

Parapoxvirus ovis strain D1701 is a highly attenuated strain that can be propagated in cell culture with titres comparable to those of the wild type virus. It has outstanding immune stimulating properties both in hosts that support replication of the infectious vector virus (e.g., sheep and goats) and in hosts that do not (e.g., dogs, swine, horse, mouse, and rat). Zylexis®, formerly known as Baypamune®, which is a preparation of chemically inactivated Parapoxvirus ovis, derived from strain D1701, is used for the prophylaxis, metaphylaxis and therapeutic treatment of infectious diseases and for preventing stress-induced diseases in animals.

The rabies virus (RV), Neurotropic lyssavirus, is a member of the Rhabdoviridae family. It is a neurotrophic virus that causes fatal disease in humans and other mammals. Rabies is most often transmitted through the bite of a rabid animal, with transmission occurring through the saliva of the animals. The vast majority of rabies cases reported to the United States Centers for Disease Control and Prevention (CDC) each year occur in wild animals like raccoons, skunks, bats, and foxes. Rabies is still a highly prevalent disease, especially in developing countries. Approximately 50,000 people die each year from rabies. The highest risk of infection for humans is from rabid dogs.

The rabies virus is a single-stranded, negative-sense RNA virus which encodes 5 proteins: nucleoprotein (N), phosphoprotein (P), matrix protein (M), glycoprotein (G), and polymerase (L). The mature bullet-shaped virus, which averages approximately 180 nm in length and 75 nm in width, has a ribonucleoprotein core, a protein coat, and a lipid envelope. Glycoprotein projections, which are about 5-10 nm long and about 3 nm in diameter, cover the outer surface of the virus. They form approximately 400 trimeric tightly arranged spikes.

RV has a high affinity for nerve tissue. The reason for this is not known. However, it may be that the rabies virus G protein can bind to the acetylcholine receptor (a neurotransmitter receptor). After the RV attaches to the host cell via the viral G protein, the virus is absorbed into the cell by engulfment.

### SUMMARY OF THE INVENTION

The present invention generally relates to recombinant Parapoxviruses, and in particular Parapoxvirus ovis (PPVO). The recombinant parapoxvirus is used for mediating a rapid innate immune response, as well as a long-lasting foreign gene-specific immunity against rabies virus. In one embodiment, a recombinant parapoxvirus comprises heterologous DNA derived from a rabies virus. In one embodiment, the recombinant parapoxvirus comprises Parapoxvirus ovis strain D1701. In one embodiment, the recombinant parapoxvirus comprises Parapoxvirus ovis strain D1701-V.

In one embodiment, the recombinant parapoxvirus comprises the gene encoding the G protein of the rabies virus, or fragments thereof. In one embodiment, the recombinant parapoxvirus comprises SEQ ID NO: 4 or a polynucleotide molecule having at least 98% identity to SEQ ID NO: 4. In one embodiment, the heterologous DNA is inserted within the HindIII fragment H/H of Parapoxvirus ovis strain D1701. In another embodiment, the heterologous DNA is inserted within the VEGF coding sequence or adjacent non-coding sequences within the HindIII fragment H/H of Parapoxvirus ovis strain D1701. In yet another embodiment, the recombinant parapoxvirus is Parapoxvirus ovis D1701-V-RabG.

The present invention embraces methods of preparing a recombinant parapoxvirus comprising inserting heterologous DNA into the genome of the parapoxvirus. In one embodiment, the method comprises the use of Parapoxvirus ovis. In one embodiment, the method comprises the use of Parapoxvirus ovis strain D1701. In one embodiment, the method comprises the use of Parapoxvirus ovis strain D1701-V. In one embodiment, the heterologous DNA used in the method comprises the gene encoding the G protein of the rabies virus, or fragments thereof. In one embodiment, the heterologous DNA used in the method comprises SEQ ID NO: 4 or a polynucleotide molecule having at least 98% identity to SEQ ID NO: 4. In one embodiment of the method, the heterologous DNA is inserted within the HindIII fragment H/H of Parapoxvirus ovis strain D1701. In another embodiment, the heterologous DNA is inserted within the VEGF coding sequence or adjacent non-coding sequences within the HindIII fragment H/H of Parapoxvirus ovis strain D1701. In one embodiment, the method comprises the preparation of Parapoxvirus ovis D1701-V-RabG. The present invention embraces an immunogenic composition comprising a recombinant parapoxvirus comprising heterologous DNA derived from a rabies virus and a carrier. The present invention embraces methods of preparing an immunogenic composition comprising combining a recombinant parapoxvirus comprising heterologous DNA derived from a rabies virus and a carrier. The present invention also embraces a vaccine comprising a recombinant parapoxvirus comprising heterologous DNA derived from a rabies virus and a carrier. The present invention embraces a method of preparing a vaccine comprising combining a recombinant parapoxvirus comprising heterologous DNA derived from a rabies virus and a carrier. In some of these embodiments, the recombinant parapoxvirus comprises Parapoxvirus ovis, while in some the recombinant parapoxvirus comprises Parapoxvirus ovis strain D1701, and in others the recombinant parapoxvirus comprises Parapoxvirus ovis strain D1701-V. In some of these embodiments, the heterologous DNA comprises the gene encoding the G protein of the rabies virus, or fragments thereof, while in other embodiments, the heterologous DNA comprises SEQ ID NO: 4 or a polynucleotide molecule having at least 98% identity to SEQ ID NO: 4. In some of these embodiments, the heterologous DNA is inserted within the HindIII fragment H/H of Parapoxvirus ovis strain D1701, in other of these embodiments, the heterologous DNA is inserted within the VEGF coding sequence or adjacent non-coding sequences within the HindIII fragment H/H of Parapoxvirus ovis strain D1701. In some of these embodiments, the recombinant parapoxvirus is Parapoxvirus ovis D1701-V-RabG.

The present invention embraces a method of inducing in an animal an immune response against rabies virus, comprising administering to said animal an immunologically effective amount of an immunogenic composition comprising a recombinant parapoxvirus comprising heterologous DNA derived from a rabies virus and a carrier. The present invention embraces a method of vaccinating an animal against rabies virus, comprising administering to said animal a therapeutically effective amount of a vaccine composition comprising the recombinant parapoxvirus comprising heterologous DNA derived from a rabies virus and a carrier. The present invention embraces a use of a recombinant parapoxvirus comprising heterologous DNA derived from a rabies virus in the preparation of a medicament for inducing an immune response against rabies virus in an animal. The present invention embraces a use of a recombinant parapoxvirus comprising heterologous DNA derived from a rabies virus in the preparation of a medicament for vaccinating an animal against rabies virus. In some of these embodiments, the recombinant parapoxvirus comprises Parapoxvirus ovis, while in some the recombinant parapoxvirus comprises Parapoxvirus ovis strain D1701, and in others the recombinant parapoxvirus comprises Parapoxvirus ovis strain D1701-V. In some of these embodiments, the heterologous DNA comprises the gene encoding the G protein of the rabies virus, or fragments thereof, while in other embodiments, the heterologous DNA comprises SEQ ID NO: 4 or a polynucleotide molecule having at least 98% identity to SEQ ID NO: 4. In some of these embodiments, the heterologous DNA is inserted within the HindIII fragment H/H of Parapoxvirus ovis strain D1701. In other of these embodiments, the heterologous DNA is inserted within the VEGF coding sequence or adjacent non-coding sequences within the HindIII fragment H/H of Parapoxvirus ovis strain D1701. In some of these embodiments, the recombinant parapoxvirus is Parapoxvirus ovis D1701-V-RabG. In some of these embodiments, an anti-G protein-specific protective immune response is induced. In other such embodiments, the immune response is the induction of anti-G protein serum antibodies. In yet other such embodiments, the induction results in antibody titers exceeding 0.5 International Units per ml.

The present invention provides methods of determining the origin of a Parapoxvirus present in an animal. The Parapoxviruses described herein can be distinguished from wild-type strains in both their genomic composition and proteins expressed. Such distinction allows for discrimination between vaccinated and infected animals. The present invention encompasses a use of a recombinant parapoxvirus as taught herein in an assay for the differentiation of infected from vaccinated animals (DIVA). In one embodiment, the recombinant Parapoxvirus ovis D1701-V-RabG is used in a DIVA assay.

These and other embodiments are disclosed and encompassed by the following detailed description.

### BRIEF DESCRIPTION OF THE DRAWINGS

A better understanding of the present invention will be gained by referring to the accompanying drawings in which:

FIG. 1: Construction of plasmid pdV-RabG. The G gene of rabies virus is inserted as a BamHI-EcoRI fragment into the multiple cloning site (boxed bases) of pdV-Rec1, which resulted in plasmid pdV-RabG (7.7 kbp in size). The position of primers ORF32N (SEQ ID NO: 1) and ORF31N (SEQ ID NO: 2) are shown. SEQ ID NO: 3 is shown in the Figure.

Abbreviations: Sm=SmaI, H3=HindIII, EcoV=EcoRV, P=PstI, B=BamHI, K=KpnI, E=EcoRI, S=SalI. F9L and ORF3 indicate the presence of the PPVO genes F9L (ORF 131) and ORF3 (ORF 133). Pvegf indicates the presence of the vegF-E promoter used to control expression of the inserted G gene. (P) and (H3) depict the destroyed PstI- and HindIII-restrictions sites of the vector backbone of plasmid pSPT-18, the T7 and SP6 promoters of pSPT-18 are indicated, respectively. The figure is not drawn to scale.

FIG. 2: Virus neutralizing serum antibody (SNT) response after immunization with different doses of D1701-V-RabG. Groups of C57/BL6 mice (for each day n=6 or 7) were immunized with a single dose of D1701-V-RabG using 10⁷ PFU (plaque-forming units) (A), 10⁶ PFU (B), 10⁵ PFU (C), and 10⁴ PFU (D). Individual sera were taken daily during 14 days after immunization (d1 to d14).

FIG. 3: Challenge experiment of immunized mice, C57/BL6 mice received a single dose of D1701-V-RabG as indicated (10⁷ to 10⁴ PFU) and were intracranially challenge infected 2 weeks later with at least 3,400 LD₅₀ (4.8×10⁵ PFU) of RV virulent strain CVS. (A) shows the survival curves of the individual animals in each group, whereas in (B) the same results were plotted in percentage of survivors.

FIG. 4. SEQ ID NO: 4—Sequence of the full length coding region of the G gene of the rabies virus (1575 nt) plus 7 nt on the 5′-end and 6 nt on the 3′-end as linker sequences for restriction enzyme analysis (BamHI and EcoRI).

FIG. 5. Role of T-cells for protective immunity. CD4-Imm is the group immunized with antisera specific for CD4 cells. CD8-Imm is the group immunized with antisera specific for CD8-T-cells. CD4/8-Imm is the group immunized with antisera specific for CD4/CD8-T-cells. Imm C is the control group vaccinated with D1701-V-RabG, which did not receive antisera. Non-Imm C is the control group that was not vaccinated with D1701-V-RabG and did not receive antisera. “Days p. chall” is the number of days post challenge with the rabies strain CVS.

FIG. 6. Protection seen with post-exposure vaccination. Non-Imm is the control group that was not vaccinated with D1701-V-RabG. D1701-V-RabG is the group that was vaccinated with D1701-V-RabG. CVS is the virulent rabies virus strain. “Days p. chall” is the number of days post challenge with the RV strain CVS.

FIG. 7. Protection seen with various post-exposure vaccination regimens. Gray squares indicate the days on which mice were vaccinated with D1701-V-RabG.

FIG. 8. Serum antibody response (determined as serum neutralization antibodies, SNT) 6 days and 13 days after immunization. Mice were immunized with D1701-V-RabG intravaginally (i.vag.), by scarification, intraperitoneally (i.p.), intradermally (i.d.), intransally (i.n.), subcutaneously (s.c.), intramuscularly (i.m.), or intraveneously (i.v.).

### DETAILED DESCRIPTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The following definitions may be applied to terms employed in the description of embodiments of the invention. They supersede any contradictory definitions contained in each individual reference incorporated herein by reference.

“About” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater, unless about is used in reference to time intervals in weeks where “about 3 weeks,” is 17 to 25 days, and about 2 to about 4 weeks is 10 to 40 days.

“Adjuvant”, as used herein, refers to any substance which serves as a non-specific stimulator of the immune response. See below for a further description of adjuvants.

The term “animal” or “animal subject”, as used herein, includes any animal that is susceptible to rabies infections, including mammals, both domesticated and wild.

“Antibody”, as used herein, is any polypeptide comprising an antigen-binding site regardless of the source, method of production, or other characteristics. It refers to an immunoglobulin molecule or a fragment thereof that specifically binds to an antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. An antibody that is “specific” for a given antigen indicates that the variable regions of the antibody recognize and bind a specific antigen exclusively. Antibodies can be a polyclonal mixture or monoclonal. Antibodies can be intact immunoglobulins derived from natural sources or from recombinant sources, or can be immunoreactive portions of intact immunoglobulins. An “antibody” can be converted to an antigen-binding protein, which includes but is not limited to antibody fragments.

The term “antigen” or “immunogen”, as used herein, refers to a molecule that contains one or more epitopes (linear, conformational or both) that upon exposure to a subject will induce an immune response that is specific for that antigen. The term “antigen” can refer to attenuated, inactivated or modified live bacteria, viruses, fungi, parasites or other microbes. The term “antigen” as used herein can also refer to a subunit antigen, which is separate and discrete from a whole organism with which the antigen is associated in nature. The term antigen also refers to antibodies, such as anti-idiotype antibodies or fragments thereof, and to synthetic peptide mimotopes that can mimic an antigen or antigenic determinant (epitope). The term “antigen” can also refer to an oligonucleotide or polynucleotide that expresses an antigen or antigenic determinant in vivo, such as in DNA immunization applications.

“Buffer” means a chemical system that prevents change in the concentration of another chemical substance, e.g., proton donor and acceptor systems serve as buffers preventing marked changes in hydrogen ion concentration (pH). A further example of a buffer is a solution containing a mixture of a weak acid and its salt (conjugate base) or a weak base and its salt (conjugate acid).

The term “cell line” or “host cell”, as used herein, means a prokaryotic or eukaryotic cell in which a virus can replicate or be maintained.

“Cellular immune response” or “cell mediated immune response” is one mediated by T-lymphocytes or other white blood cells or both, and includes the production of cytokines, chemokines and similar molecules produced by activated T-cells, white blood cells, or both.

“Conservative substitution” is defined in the art and known to one skilled in the art, and is recognized to classify residues according to their related physical properties.

The term “culture”, as used herein, means a population of cells or microorganisms growing in the absence of other species or types.

The term “DIVA” as used herein means to Differentiate Infected from Vaccinated Animals.

“Dose” refers to a vaccine or immunogenic composition given to a subject. A “first dose” or “priming vaccine” refers to the dose of such a composition given on Day 0. A “second dose” or a “third dose” or an “annual dose” refers to an amount of such composition given subsequent to the first dose, which may or may not be the same vaccine or immunogenic composition as the first dose.

An “epitope” is the specific site of the antigen which binds to a T-cell receptor or specific antibody, and typically comprises from about 3 amino acid residues to about 20 amino acid residues.

“Excipient” refers to any component of a vaccine or immunogenic composition that is not an antigen.

“Fragment” refers to a truncated portion of a protein or gene. “Functional fragment” and “biologically active fragment” refer to a fragment that retains the biological properties of the full length protein or gene. An “immunogenically active fragment” refers to a fragment that elicits an immune response.

The term “G protein”, as used herein, refers to protein in the glycoprotein projections that cover the outer surface of a rabies virus.

The term “heterologous”, as used herein, means derived from a different species or strain.

The term “homologous”, as used herein, means derived from the same species or strain.

“Homology” or “percent homology” refers to the percentage of nucleotide or amino acid residues in the candidate sequence that are identical with the residues in the comparator sequences after aligning the sequences and introducing gaps, if necessary, to achieve the maximum percent sequence identity, and also considering any conservative substitutions as part of the sequence identity.

“Humoral immune response” refers to one that is at least in part mediated by antibodies.

“Identity” or “percent identity” refers to the percentage of nucleotides or amino acids in the candidate sequence that are identical with the residues in the comparator sequence after aligning both sequences and introducing gaps, if necessary, to achieve the maximum percent sequence identity, and not considering any conservative substitutions as part of the sequence identity.

“Immune response” in a subject refers to the development of a humoral immune response, a cellular immune response, or a humoral and a cellular immune response to an antigen. The immunogenic response may be sufficient for diagnostic purposes or other testing, or may be adequate to prevent signs or symptoms of disease, including adverse health effects or complications thereof, caused by infection with a disease agent. Immune responses can usually be determined using standard immunoassays and neutralization assays, which are known in the art.

“Immunogenic” or “Immunogenicity”, as used herein, refers to the capability to elicit an immune response directed specifically against an antigen.

The terms “immunogenic composition,” or “immunologically effective amount,” or “amount effective to produce an immune response,” as used herein, refer to a composition or antigen capable of being recognized by the immune system, resulting in the generation of a specific immune response (i.e., has immunogenic activity) when administered alone or with a pharmaceutically acceptable carrier, to an animal.

“Isolated”, as used herein, means removed from its naturally occurring environment, either alone or in a heterologous host cell, or chromosome or vector (e.g., plasmid, phage, etc.). “Isolated bacteria,” “isolated anaerobic bacteria,” “isolated bacterial strain,” “isolated virus” “isolated viral strain” and the like refer to a composition in which the bacteria or virus are substantial free of other microorganisms, e.g., in a culture, such as when separated from it naturally occurring environment. “Isolated,” when used to describe any particularly defined substance, such as a polynucleotide or a polypeptide, refers to the substance that is separate from the original cellular environment in which the substance—such as a polypeptide or nucleic acid—is normally found. As used herein therefore, by way of example only, a recombinant cell line constructed with a polynucleotide of the invention makes use of the “isolated” nucleic acid. Alternatively, if a particular protein or a specific immunogenic fragment is claimed or used as a vaccine or other composition, it would be considered to be isolated because it had been identified, separated and to some extent purified as compared to how it may exist in nature. If the protein or a specific immunogenic fragment thereof is produced in a recombinant bacterium or eukaryote expression vector that produces the antigen, it is considered to exist as an isolated protein or nucleic acid. For example, a recombinant cell line constructed with a polynucleotide makes use of an “isolated” nucleic acid.

“Medicinal agent” refers to any agent which is useful in the prevention, cure, or improvement of disease, or the prevention of some physiological condition or occurrence.

The term “multiplicity of infection” (MOI) refers to a ratio of the number of organisms per cell, which details how much inoculum is going to be used in a given infection.

The terms “parapoxvirus”, “parapoxvirus strains”, as used herein, refer to viruses belonging to the family Poxyiridae and the genus Parapoxvirus.

The terms “Parapoxvirus ovis” and “Parapoxvirus ORFV”, as used herein, refer to viruses belonging to the family Poxyiridae, the genus Parapoxvirus, and the species Parapoxvirus ovis. These viruses are also called ecthyma contagiosum virus, contagious pustular dermatitis virus, or orf virus. They possess a unique spiral coat that distinguishes them from the other poxviruses.

The term “Parapoxvirus ovis strain D1701” refers to the virus as described in U.S. Pat. No. 6,365,393, which is incorporated herein by reference. “Parapoxvirus ovis strain D1701-V” refers to Parapoxvirus ovis strain D1701 adapted to the simian cell line Vero.

“Parenteral administration” refers to the introduction of a substance, such as a vaccine, into a subject's body through or by way of a route that does not include the digestive tract. Parenteral administration includes subcutaneous, intramuscular, transcutaneous, intradermal, intraperitoneal, intraocular, and intravenous administration.

The term “pathogen” or “pathogenic microorganism”, as used herein, means a microorganism—for example a rabies virus—which is capable of inducing or causing a disease, illness, or abnormal state in its host animal.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

The term “poxvirus”, as used herein, refers to viruses belonging to the family Poxyiridae. These viruses are oval, quite large, double-stranded DNA viruses.

The term “polynucleotide” or “polynucleotide molecule”, as used herein, means an organic polymer molecule composed of nucleotide monomers covalently bonded in a chain. DNA (deoxyribonucleic acid) and RNA (ribonucleic acid) are examples of polynucleotides with distinct biological function.

The terms “prevent”, “preventing” or “prevention”, and the like, as used herein, mean to inhibit the replication of a microorganism, to inhibit transmission of a microorganism, or to inhibit a microorganism from establishing itself in its host. These terms and the like as used herein can also mean to inhibit or block one or more signs or symptoms of infection. The treatment is considered therapeutic if there is a reduction in the microorganism load.

“Protection”, “protecting”, and the like, as used herein with respect to a vaccine or other composition, means that the vaccine or composition prevents or reduces the symptoms of the disease caused by the organism from which the antigen(s) used in the vaccine or composition is derived. The terms “protection” and “protecting” and the like, also mean that the vaccine or composition can be used to therapeutically treat the disease or one of more symptoms of the disease that already exists in a subject.

The term “rabies virus” refers to Neurotropic lyssavirus, a member of the Rhabdoviridae family. It is a single-stranded, negative-sense RNA virus which has glycoprotein projections on its outer surface.

“Recombinant PPV” or “Recombinant PPV” are PPV having insertions and/or deletions in their genome. The insertions and deletions are prepared using molecular biological methods.

“Species homologs” include genes found in two or more different species which possess substantial polynucleotide sequence homology and possess the same, or similar, biological functions and/or properties. Preferably polynucleotide sequences which represent species homologs will hybridize under moderately stringent conditions, as described herein by example, and possess the same or similar biological activities and or properties. In another aspect, polynucleotides representing species homologs will share greater than about 60% sequence homology, greater than about 70% sequence homology, greater than about 80% sequence homology, greater than about 90% sequence homology, greater than about 95% sequence homology, greater than about 96% sequence homology, greater than about 97% sequence homology, greater than about 98% sequence homology, or greater than about 99% sequence homology.

The terms “specific binding,” “specifically binds,” and the like, are defined as two or more molecules that form a complex that is measurable under physiologic or assay conditions and is selective. An antibody or other inhibitor is said to “specifically bind” to a protein if, under appropriately selected conditions, such binding is not substantially inhibited, while at the same time non-specific binding is inhibited. Specific binding is characterized by high affinity and is selective for the compound or protein. Nonspecific binding usually has low affinity. Binding in IgG antibodies, for example, is generally characterized by an affinity of at least about 10⁻⁷ M or higher, such as at least about 10⁻⁸ M or higher, or at least about 10⁻⁹ M or higher, or at least about 10⁻¹⁰ or higher, or at least about 10⁻¹¹ M or higher, or at least about 10⁻¹² M or higher. The term is also applicable where, e.g., an antigen-binding domain is specific for a particular epitope that is not carried by numerous antigens, in which case the antibody carrying the antigen-binding domain will generally not bind other antigens.

“Specific immunogenic fragment”, as used herein, refers to a portion of a sequence that is recognizable by an antibody or T cell specific for that sequence.

“Subject” refers to any animal that is susceptible to rabies infections, including mammals, both domesticated and wild.

“Substantially identical”, as used herein, refers to a degree of sequence identity of at least about 90%, at least about 95%, at least about 96%, at least about 97%, at least about 98%, or at least about 99%.

“Therapeutically effective amount”, as used herein, refers to an amount of an antigen or vaccine or composition that would induce an immune response in a subject (e.g., dog) receiving the antigen or vaccine or composition which is adequate to prevent or ameliorate signs or symptoms of disease, including adverse health effects or complications thereof, caused by infection with a pathogen, such as a virus, bacterium, parasite or fungus. Humoral immunity or cell-mediated immunity, or both humoral and cell-mediated immunity, can be induced. The immunogenic response of an animal to an antigen, vaccine, or composition can be evaluated indirectly through measurement of antibody titers, lymphocyte proliferation assays, or directly through monitoring signs and symptoms after challenge with the wild type strain. The protective immunity conferred by a vaccine or composition can be evaluated by measuring reduction of challenge organism shed, and/or reduction in clinical signs, such as mortality, morbidity, temperature, and overall physical condition, health, and performance of the subject. The amount of a vaccine or composition that is therapeutically effective can vary, depending on the particular immunogen used, or the condition of the subject, and can be determined by one skilled in the art.

The terms “treat”, “treating” or “treatment”, and the like, as used herein, mean to reduce or eliminate an infection by a microorganism. These terms and the like can also mean to reduce the replication of a microorganism, to reduce the transmission of a microorganism, or to reduce the ability of a microorganism to establish itself in its host. These terms and the like as used herein can also mean to reduce, ameliorate, or eliminate one or more signs or symptoms of infection by a microorganism, or accelerate the recovery from infection by a microorganism.

The terms “vaccinate” and “vaccinating” and the like, as used herein, mean to administer to an animal a vaccine or immunogenic composition.

The terms “vaccine” and “vaccine composition,” as used herein, mean a composition which prevents or reduces an infection, or which prevents or reduces one or more signs or symptoms of infection. The protective effects of a vaccine composition against a pathogen are normally achieved by inducing in the subject an immune response. Generally speaking, abolished or reduced incidences of infection, amelioration of the signs or symptoms, or accelerated elimination of the microorganism from the infected subjects are indicative of the protective effects of a vaccine composition. The vaccine compositions of the present invention provide protective effects against infections caused by rabies virus.

The term “variant,” as used herein, refers to a derivation of a given protein and/or gene sequence, wherein the derived sequence is essentially the same as the given sequence, but for mutational differences. Said differences may be naturally-occurring, or synthetically- or genetically-generated.

A “vector” or a “vector virus” is a PPV which is suitable for the insertion of heterologous DNA, which can transport the inserted DNA into cells or organisms, and which, where appropriate, enables the heterologous DNA to be expressed.

The term “veterinarily acceptable carrier” as used herein refers to substances, which are within the scope of sound medical judgment, suitable for use in contact with the tissues of animals without undue toxicity, irritation, allergic response, and the like, commensurate with a reasonable benefit-to-risk ratio, and effective for their intended use.

The following description is provided to aid those skilled in the art in practicing the present invention. Even so, this description should not be construed to unduly limit the present invention as modifications and variations in the embodiments discussed herein can be made by those of ordinary skill in the art without departing from the spirit or scope of the present inventive discovery.

### Viruses, Immunogenic Compositions, and Vaccines

The present invention embraces the use of parapoxviruses for the preparation of a recombinant parapoxvirus comprising heterologous DNA derived from a rabies virus.

In one embodiment, Parapoxvirus ovis (PPVO) for the preparation of a recombinant parapoxvirus comprising heterologous DNA derived from a rabies virus is used. In another embodiment, the Parapoxvirus ovis strain D1701 is used. In a further embodiment, the Parapoxvirus ovis strain D1701-V is used.

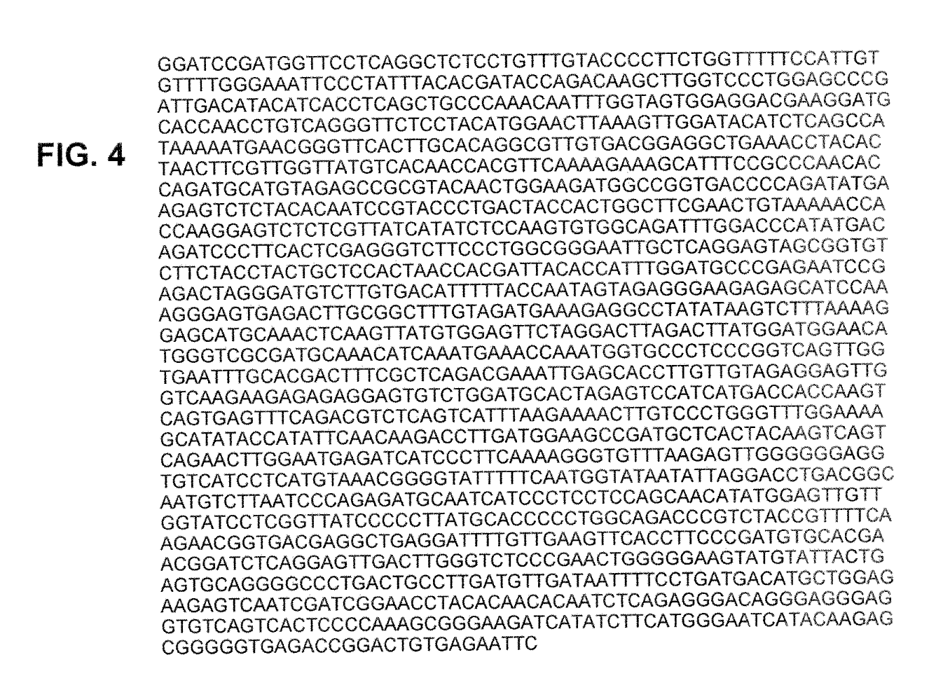

The genetic sequence inserted into the parapoxvirus includes heterologous DNA derived from a rabies virus. In one embodiment, the heterologous DNA comprises the gene encoding the G protein of the rabies virus, or fragments thereof. In one embodiment, the heterologous DNA comprises SEQ ID NO: 4 or a polynucleotide molecule having at least 98% identity to SEQ ID NO: 4. The structure of this gene is further disclosed, for example, by Anilionis et al., Nature 294, 275-278 (1981). The insert is 1588 nt in size. The complete sequence of the insert (SEQ ID NO: 4) is shown in FIG. 4. It contains the full-length coding region of the 0 gene of rabies virus (1575 nt) plus 7 nt on the 5′-end and 6 nt on the 3′-end as linker sequences for restriction enzyme analysis (BamHI and EcoRI).

Knowledge of the sequence of a polynucleotide makes readily available every possible fragment of that polynucleotide. The invention therefore provides for fragments of the G protein. In one embodiment, functional fragments are provided for. In another embodiment, biologically active fragments are provided for. Fragments can be purified by conventional methods, such as for example by filtration or chromatography. Fragments can be produced by recombination by methods known to one skilled in the arts.

In preparing the recombinant parapoxvirus, the heterologous DNA is inserted within the HindIII fragment H/H of Parapoxvirus ovis strain D1701. In another embodiment, the heterologous DNA is inserted in within the VEGF coding sequence or adjacent non-coding sequences within the HindIII fragment H/H of Parapoxvirus ovis strain D1701. The methods used to insert the heterologous DNA into the parapoxvirus are standard and known to one skilled in the art. They are described in U.S. Pat. No. 6,365,393.

In one embodiment, the recombinant parapoxvirus comprising heterologous DNA derived from a rabies virus is Parapoxvirus ovis D1701-V-RabG.

In one embodiment, the recombinant parapoxvirus comprising heterologous DNA derived from a rabies virus is D1701-VrV RabG (K-UC1002), which is deposited at The American Type Culture Collection (ATCC®), Manassas, Va., 20108 USA with ATCC® Patent Deposit Designation PTA-11662, in compliance with Budapest Treaty on the International Recognition of the Deposit of Microorganisms for the Purposes of Patent Procedure.

The sequence of the plasmid pdV-RabG (7.692 nt) is SEQ ID NO: 5, which is shown in the sequence listing.

The invention also embraces polynucleotide sequences that have at least about 99%, at least about 98%, at least about 97%, at least about 96%, at least about 95%, at least about 93%, at least about 90%, at least about 85%, at least about 80%, at least about 75%, at least about 70%, at least about 65%, at least about 60%, at least about 55%, and at least about 50% identity and/or homology to the sequences described herein.

The invention also embraces polynucleotide sequences which hybridize under moderately to highly stringent conditions to the non-coding strand, or complement, of any one of the SEQ ID NOs described herein, and species homologs thereof. Exemplary high stringency conditions include a final wash in buffer comprising 0.2× SSC/0.1% SDS, at 65° C. to 75° C., while exemplary moderate stringency conditions include a final wash in buffer comprising 2×SSC/0.1% SDS, at 35° C. to 45° C. It is understood in the art that conditions of equivalent stringency can be achieved through variation of temperature and buffer, or salt concentration as described in Ausubel, et al. (Eds.), Protocols in Molecular Biology, John Wiley & Sons (1994), pp. 6.0.3 to 6.4.10.

The recombinant PPV can be propagated in cells, cell lines and host cells. Said cells, cell lines, or host cells may be for example, but are not limited to, mammalian cells and non-mammalian cells. Cells, cell lines, and host cells in which the PPV can be propagated are readily known and accessible to those of ordinary skill in the art. In one embodiment Vero cells are used. In other embodiments, bovine kidney or ovine testis cells are used.

The recombinant PPV can be further attenuated or inactivated prior to use in an immunogenic composition or vaccine. Methods of attenuation and inactivation are well known to those skilled in the art. Methods for attenuation include, but are not limited to, serial passage in cell culture on a suitable cell line, ultraviolet irradiation, and chemical mutagenesis. Methods for inactivation include, but are not limited to, treatment with formalin, betapropriolactone (BPL) or binary ethyleneimine (BEI), or other methods known to those skilled in the art.

Inactivation by formalin can be performed by mixing the virus suspension with 37% formaldehyde to a final formaldehyde concentration of 0.05%. The virus-formaldehyde mixture is mixed by constant stirring for approximately 24 hours at room temperature. The inactivated virus mixture is then tested for residual live virus by assaying for growth on a suitable cell line.

Inactivation by BEI can be performed by mixing the virus suspension of the present invention with 0.1 M BEI (2-bromo-ethylamine in 0.175 N NaOH) to a final BEI concentration of 1 mM. The virus-BEI mixture is mixed by constant stirring for approximately 48 hours at room temperature, followed by the addition of 1.0 M sodium thiosulfate to a final concentration of 0.1 mM. Mixing is continued for an additional two hours. The inactivated virus mixture is tested for residual live virus by assaying for growth on a suitable cell line.

The recombinant PPV can be used in immunogenic compositions and vaccines.

The immunogenic compositions and vaccines optionally can include one or more veterinarily acceptable carriers, including liquid, semisolid, or solid diluents, that serve as pharmaceutical vehicles, excipients, or media. As used herein, a “veterinarily-acceptable carrier” includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others known to those skilled in the art. Stabilizers include albumin, among others known to the skilled artisan. Preservatives include merthiolate, among others known to the skilled artisan.

Adjuvants include, but are not limited to, the RIBI adjuvant system (Ribi Inc.), alum, aluminum hydroxide gel, oil-in water emulsions, water-in-oil emulsions such as, e.g., Freund's complete and incomplete adjuvants, Block co polymer (CytRx, Atlanta Ga.), SAF-M (Chiron, Emeryville Calif.), AMPHIGEN® adjuvant, saponin, Quil A, QS-21 (Cambridge Biotech Inc., Cambridge Mass.), GPI-0100 (Galenica Pharmaceuticals, Inc., Birmingham, Ala.) or other saponin fractions, monophosphoryl lipid A, Avridine lipid-amine adjuvant, heat-labile enterotoxin from E. coli (recombinant or otherwise), cholera toxin, or muramyl dipeptide, among many others known to those skilled in the art. The amounts and concentrations of adjuvants and additives useful in the context of the present invention can readily be determined by the skilled artisan. In one embodiment, the present invention contemplates immunogenic compositions and vaccines comprising from about 50 μg to about 2000 μg of adjuvant. In another embodiment adjuvant is included in an amount from about 100 μg to about 1500 μg, or from about 250 μg to about 1000 μg, or from about 350 μg to about 750 μg. In another embodiment, adjuvant is included in an amount of about 500 μg/2 ml dose of the immunogenic composition or vaccine.

The immunogenic compositions and vaccines can also include antibiotics. Such antibiotics include, but are not limited to, those from the classes of aminoglycosides, carbapenems, cephalosporins, glycopeptides, macrolides, penicillins, polypeptides, quinolones, sulfonamides, and tetracyclines. In one embodiment, the present invention contemplates immunogenic compositions and vaccines comprising from about 1 μg/ml to about 60 μg/ml of antibiotic. In another embodiment, the immunogenic compositions and vaccines comprise from about 5 μg/ml to about 55 μg/ml of antibiotic, or from about 10 μg/ml to about 50 μg/ml of antibiotic, or from about 15 μg/ml to about 45 μg/ml of antibiotic, or from about 20 μg/ml to about 40 μg/ml of antibiotic, or from about 25 μg/ml to about 35 μg/ml of antibiotic. In yet another embodiment, the immunogenic compositions and vaccines comprise less than about 30 μg/ml of antibiotic.

In addition to the recombinant PPV, immunogenic compositions and vaccines can include other antigens. Antigens can be in the form of an inactivated whole or partial preparation of the microorganism, or in the form of antigenic molecules obtained by genetic engineering techniques or chemical synthesis. Other antigens appropriate for use in accordance with the present invention include, but are not limited to, those derived from pathogenic bacteria or pathogenic viruses.

In the case of dogs, the recombinant rabies immunogenic compositions and vaccines can also optionally contain a mixture with one or more additional canine antigens such as, for example, Ehrlichia canis, canine parvovirus (CPV), canine distemper, canine parainfluenza virus (CPI), canine adenovirus type II (CAV-2), canine adenovirus (CDV), canine coronavirus (CCV), Leptospira icterohemorrhagiae (LI), Leptospira canicola (LC), Leptospira grippotyphosa (LG), Leptospira pomona (LP), Borrelia burgdorferi, and the like. One combination of antigens encompasses isolates of canine Parvovirus, canine distemper, canine adenovirus and canine parainfluenza, with or without coronavirus and Leptospira (including the emerging serovars Leptospira grippotyphosa and Leptospira pomona).

In the case of cats, the recombinant rabies immunogenic compositions and vaccines can also optionally contain a mixture with one or more additional feline antigens such as, for example, feline calicivirus (FCV), Chlamydophila felis (C. felis, also previously and commonly known as Chlamydia psittaci (FCP)), feline leukemia virus (FeLV), feline panleukopenia virus (FPV), feline rhinotracheitis virus (FVR), feline immunodeficiency virus (Fly), feline infectious peritonitis virus (FIPV), Bartonella henselae (e.g., cat scratch disease) and the like.

In the case of horses, the recombinant rabies immunogenic compositions and vaccines can also optionally contain a mixture with one or more additional equine antigens such as, for example, Equine influenza virus, Equine herpesvirus 1 and 4, Equine arterivirus, West Nile virus, Equine rotavirus, Streptococcus equi, Tetanus toxoid, and the like.

Immunogenic compositions and vaccines described herein can be administered to an animal to induce an effective immune response against RV. Accordingly, described herein are methods of stimulating an effective immune response against RV comprising administering to an animal a therapeutically effective amount of an immunogenic composition or vaccine comprising a recombinant parapoxvirus comprising heterologous DNA derived from a rabies virus. The method results in the induction of anti-G protein serum antibodies.

Immunogenic compositions and vaccines described herein can be administered to an animal to vaccinate the animal against rabies disease. The immunogenic compositions and vaccines can be administered to the animal to prevent or treat rabies disease in the animal. Accordingly, described herein are methods of vaccinating an animal against rabies disease, and preventing or treating rabies disease, comprising administering to the animal a therapeutically effective amount of an immunogenic composition or vaccine comprising a recombinant parapoxvirus comprising heterologous DNA derived from a rabies virus.

### Forms, Dosages, Routes of Administration

Immunogenic compositions and vaccines can be made in various forms depending upon the route of administration. For example, the immunogenic compositions and vaccines can be made in the form of sterile aqueous solutions or dispersions suitable for injectable use, or made in lyophilized forms using freeze-drying techniques. Lyophilized immunogenic compositions and vaccines are typically maintained at about 4° C., and can be reconstituted in a stabilizing solution, e.g., saline or and HEPES. Alternatively, immunogenic compositions and vaccines can be preserved by freeze drying. Immunogenic compositions and vaccines can also be made in the form of suspensions or emulsions.

Immunogenic compositions and vaccines include a therapeutically effective amount of the above-described recombinant PPV. Purified viruses can be used directly in an immunogenic composition or vaccine, or can be further attenuated, or inactivated, Typically, an immunogenic composition or vaccine contains between about 1×10² and about 1×10¹² PFU, or between about 1×10³ and about 1×10¹¹ PFU, or between about 1×10⁴ and about 1×10¹⁰ PFU, or between about 1×10⁵ and about 1×10⁹ PFU, or between about 1×10⁶ and about 1×10⁸ PFU. The precise amount of a virus in an immunogenic composition or vaccine effective to provide a protective effect can be determined by a skilled artisan.

The immunogenic compositions and vaccines generally comprise a veterinarily acceptable carrier in a volume of between about 0.5 ml and about 5 ml. In another embodiment the volume of the carrier is between about 1 ml and about 4 ml, or between about 2 ml and about 3 ml. In another embodiment, the volume of the carrier is about 1 ml, or is about 2 ml, or is about 3 ml, or is about 5 ml. Veterinarily acceptable carriers suitable for use in immunogenic compositions and vaccines can be any of those described herein.

Those skilled in the art can readily determine whether a virus needs to be attenuated or inactivated before administration. In another embodiment, the recombinant PPV can be administered directly to an animal without additional attenuation. The amount of a virus that is therapeutically effective can vary depending on any of several factors including the condition of the animal and the degree of infection, and can be determined by a skilled artisan.

In accordance with the methods of the present invention, a single dose can be administered to animals, or, alternatively, two or more inoculations can take place with intervals of from about two to about ten weeks. Boosting regimens can be required and the dosage regimen can be adjusted to provide optimal immunization. Those skilled in the art can readily determine the optimal administration regimen.

Immunogenic compositions and vaccines can be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which can contain excipients such as salts, carbohydrates and buffering agents (preferably to a pH of from about 3 to about 9, or from about 4 to about 8, or from about 5 to about 7.5, or from about 6 to about 7.5, or about 7 to about 7.5), but, for some applications, they can be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, can readily be accomplished using standard pharmaceutical techniques well known to those skilled in the art.

The recombinant parapoxviruses and immunogenic compositions and vaccines described herein can be used in the preparation of a medicament for vaccinating an animal against rabies disease.

The present invention provides methods of determining the origin of a parapoxvirus present in an animal.

Vaccination which utilizes a DIVA vaccine—one which is able to differentiate infected from vaccinated animals—provides a means for determining the origin of a parapoxvirus present in an animal. This differentiation can be accomplished via any of various diagnostic methods, including but not limited to ELISA, Western blotting and PCR. These and other methods are readily recognized and known to one of ordinary skill in the art.

The parapoxviruses described herein can be distinguished from wild-type strains in both their genomic composition and proteins expressed. Such distinction allows for discrimination between vaccinated and infected animals. For example, a determination can be made as to whether an animal testing positive for parapoxvirus in certain laboratory tests carries a wild-type parapoxvirus strain, or carries a recombinantly produced parapoxvirus previously obtained through vaccination.

A variety of assays can be employed for making the determination. For example, virus can be isolated from the animal testing positive for parapoxvirus, and nucleic acid-based assays can be used to determine the presence of a parapoxvirus genome, indicative of prior vaccination. The nucleic acid-based assays include Southern or Northern blot analysis, PCR, and sequencing. Alternatively, protein-based assays can be employed. In protein-based assays, cells or tissues suspected of an infection can be isolated from the animal testing positive for parapoxvirus. Cellular extracts can be made from such cells or tissues and can be subjected to, e.g., Western Blot, using appropriate antibodies against viral proteins that can distinctively identify the presence of either the recombinantly produced parapoxvirus previously inoculated, or wild-type parapoxvirus.

The extent and nature of the immune responses induced in the animal can be assessed by using a variety of techniques. For example, sera can be collected from the inoculated animals and tested for the presence or absence of antibodies specific for the parapoxvirus e.g. in a conventional ELISA. Detection of responding cytotoxic T-lymphocytes (CTLs) in lymphoid tissues can be achieved by assays such as T cell proliferation, as indicative of the induction of a cellular immune response. The relevant techniques are well described in the art, e.g., Coligan et al. Current Protocols in Immunology, John Wiley & Sons Inc. (1994).

The recombinant Parapoxvirus ovis D1701-V-RabG can be used in a DIVA assay. In one embodiment, it can be used in assays for the detection of rabies N-genes or proteins to differentiate infected from vaccinated animals. In another embodiment, it can be used in assays for the detection of rabies P-genes or proteins to differentiate infected from vaccinated animals. In yet another embodiment, it can be used in assays for the detection of rabies L-genes or proteins to differentiate infected from vaccinated animals. In still another embodiment, it can be used in assays for the detection of rabies M-genes or proteins to differentiate infected from vaccinated animals.

The present invention is additionally described by the following illustrative, non-limiting Examples.

### EXAMPLES

A recombinant Parapoxvirus ovis, which comprises the gene encoding for the G protein of RV, was generated. Expression of the G protein was assessed, as was the immunostimulatory and protective properties of the recombinant virus against RV.

### Example 1

### Generation of Rabies G Protein Expressing Recombinant Virus D1701-V-RabG

A recombinantly-generated Parapoxvirus ovis, which contains the gene encoding for the G protein of RV, was generated. Expression of the G protein was assessed, as was the immunostimulatory and protective properties of the recombinant virus against RV.

### Construction of Transfer Plasmid.

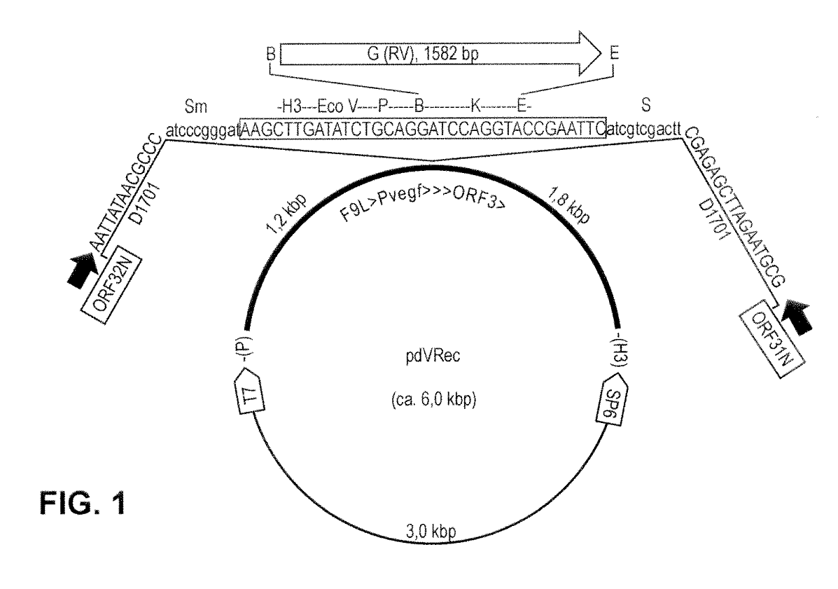

For generation of the recombinant Parapoxvirus ovis D1701-V-RabG, the Parapoxvirus ovis (PPVO) vector system (U.S. Pat. No. 6,365,393; Rziha et al., 2000, J. Biotechnol., 83, 137-145; Fischer et al., 2003, J. Virol. 77, 9312-9323; Henkel et al., 2005, J. Viral. 79, 314-325) was used. The Rabies virus G protein gene was chemically synthesized (Blue Heron Biotech; USA) and provided in a pUC plasmid. The complete G gene was isolated as a BamHI-EcoRI DNA fragment of 1.582 bp in size by agarose gel (0.8% w/v) electrophoresis and purified by Qiaex II gel extraction kit (Qiagen; Germany). The plasmid pdV-Rec1 (Fischer et al., 2003) was double-digested with BamHI and EcoRI and used for ligation (Fast ligation kit, Promega; Germany). After transformation of E. coli DH5αF′ (Invitrogen, Thermo Fisher Scientific; Germany) insert-positive colonies were selected by EcoRI-BamHI restriction digestion of plasmid DNA. This resulted in transfer plasmid pdV-RabG (FIG. 1), which were prepared by Qiagen Plasmid Maxi Kit (Qiagen; Germany) and used for DNA sequencing. To this end, primer ORF32N (SEQ ID NO: 1; 5′-GCGCGCTGCGGGTGCGCTACCAATTCGCGC-3′) located upstream of the insertion site and primer ORF31N (SEQ ID NO: 2; 5′-GCATCCCGTTACCACCGGAGACCGACGCTCCC-3′) located downstream of the insertion site in pdV-Rec1 was used as well as internal G-gene-specific primers RabG-F (SEQ ID NO: 6; 5′-GGAGTCTCTCGTTATCATATCTC-3′) and RabG-R (SEQ ID NO: 7; 5′-CTAAACAAGGTGCTCAATTTCGT-3′), respectively. This allowed the determination of the complete sequence of the inserted G gene (SEQ ID NO: 4).

### Selection of Recombinants by Bluo-Gal Staining.

Vero cells (10⁶ cells) were infected with 0.1 MOI (multiplicity of infection) of the lacZ-expressing virus D1701-VrV, and 2 hr later transfected with 2 μg of pdV-RabG plasmid DNA by nucleofection, according to the manufacturer's recommendation (Amaxa Nucleofector, Lonza; Germany). Virus lysates were harvested 3-4 days later, and used for titration on Vero cells in 6-well plates (Fisher Scientific; Germany). When plaques became visible, agarose-containing Bluo-Gal was overlaid as described (Fischer et al., 2003). Virus plaques having a white appearance were picked, and the single plaque eluates (overnight at 4° C. in phosphate-buffered saline (PBS)) were used for simultaneous infection of Vero cells (1×10⁵ cells) in single wells of a 48-well plate.

### Selection of Recombinants by Plaque-PCR.

Isolation of DNA from each virus plaque isolate was performed in a modification of Pasamontes et al. (J. Virol. Methods 35:137-141; 1991). Virus lysate (0.2 ml) was frozen (−70° C.) and thawed (37° C.) three times, and sonicated on ice 3 times for 20-30 sec (sonic waterbath). After phenol and chloroform extraction, 10 μg yeast tRNA or 3 μl GlycoBlue (Ambion; Germany) were added before ethanol precipitation. The DNA pellet was washed twice with 70% (v/v) ethanol, and dissolved after drying in 14 μl aqua bidest.

For RabG-specific PCR, 4 μl of the DNA were mixed on ice with 1 μl primer mix, consisting of 4.0 pmol RabG-F (SEQ ID NO: 6) and 4.0 pmol RabG-R (SEQ ID NO: 7) primers, and 5 μl ReddyMix 2×PCR (Abgene, Thermo Fisher Scientific; Germany). PCR was performed in a Trio Thermoblock (Biometra; Germany) by incubating for 2 min at 98° C., followed by 40 cycles for 1 min at 96° C., 30 sec at 60° C., 30 sec at 72° C., and a final extension step for 2 min at 72° C. The PCR products were separated in horizontal 1% (w/v) agarose-ethidiumbromide (0.3 microgram per ml) gels. Virus lysates from plaque isolates revealing the G gene-specific PCR fragment of 433 bp in size were diluted and further plaque-purified at least 3 times, using Bluo-Gal agarose overlay as described above. Finally, the DNA of recombinant virus plaque isolates positive for the G gene were tested in a LacZ gene-specific PCR using 4 μl DNA, 3.95 pmol primer lacZ-F (SEQ ID NO: 8; 5′-CGATACTGTCGTCGTCCCCTCAA-31, and 4.13 pmol primer lacZ-R (SEQ ID NO: 9; 5′-CAACTCGCCGCACATCTGAACT-3′). After adding 5 μl AccuPrime SuperMix II (Invitrogen, Fisher Scientific; Germany), PCR was performed by heating for 2 min at 98° C., followed by 40 cycles for 1 min at 96° C., 30 sec at 62° C., and 90 sec at 68° C., with a final extension step for 2 min at 68° C. Separation of PCR products was performed as described above. The absence of the LacZ gene-specific fragment of 508 bp in size demonstrated that the corresponding recombinant virus plaque isolates were free of the lacZ-expressing parental virus D1701-VrV following three rounds of plaque purification. After preparation of high titer virus stocks of D1701-V-RabG, viral DNA was prepared as described below, and used for testing in RabG-PCR and LacZ-PCR,

### Immunohistochemical Staining of Recombinant Virus Plaques (IPMA).

Successful expression of inserted foreign gene was first assayed by IPMA, which involves immunohistochemical staining of recombinant virus plaques titrated on Vero cells in 24-well plates. After the appearance of virus plaques, the medium was aspirated from each well, and the cells dried by leaving the plate open for approximately 10 min in a laminar flow hood. Thereafter, the cells were fixed with ice-cold absolute methanol at −20° C. for 15-20 min. After washing twice with ice-cold 1% (v/v) fetal calf serum (FCS) in PBS, the cells were blocked with PBS containing 10% (v/v) FCS for 90 min at room temperature (RT). After incubation for 60 min at RT with the G protein-specific mouse monoclonal antibody G559, diluted 1:200 in 1% FCS in PBS (FLI-Tuebingen; Germany). After 3 washes with PBS-T (PBS containing 0.05% (v/v) Tween-20), a peroxidase-coupled anti-mouse secondary antibody (Jackson-ImmunoRes., DIANOVA; Germany), diluted 1:2000, was added, and incubated for 60 min at RT. After thorough washing with PBS-T and PBS, substrate (Vector Nova Red, Axxora; Germany) was added as recommended by the manufacturer's instruction, until red-brown positive staining became visible. As negative controls, non-infected cells and cells infected with D-1701-VrV or D-1701-V were included. Virus plaques and infected cells were found strongly positive for Rabies virus G protein.

### Example 2

### Characterization of D1701-V-RabG

### Preparation of Virus Stocks.

To obtain high titer recombinant virus stocks, 10-20 T150 culture flasks (Greiner; Germany) were simultaneously infected with a MOI of 0.5. After 3 days, approximately 80% cytopathogenic effect (CPE) was observed, and the cells and supernatant of all flasks were harvested and collected for centrifugation (2 hr at 13,000 rpm, 4° C.). The supernatant was carefully removed, and the virus pellet was dissolved overnight at 4° C. in 1-2 ml PBS. The virus suspension was completely dispersed by sonification (Sonic cell disruptor, Branson; Germany) on ice using 3 pulses (100 W) of 10 sec, (10 sec break between each pulse), followed by centrifugation (500-700×g, 10 min, 4° C.) to remove cell debris. The supernatant was stored on ice, while the cell pellet was resuspended in 1.0 ml PBS, and sonicated on ice (2 times for 20 sec, with a 10 sec break in between, then once for 30 sec). After low speed centrifugation, the supernatant was combined with the first supernatant, divided into aliquots, titrated, and stored at −70° C.

### Characterization of Viral DNA.

Vero cells were infected with MOI 0.5, and harvested after 2-3 days (approx. 80% CPE) by trypsinisation and brief low speed centrifugation at 4° C. DNA was isolated using the Master Pure DNA Isolation Kit (Epicentre Biotechnology, Biozym Scientific; Germany), according to the manufacturer's protocol.

To verify insertion of the G gene in the correct locus, 2 μg DNA were restriction enzyme-digested, separated in 0.8% (w/v) agarose gels, and transferred to nylon membrane (GE Healthcare; Germany) for Southern blot hybridization according to standard procedures. A Rabies G gene-specific probe (the product of the Rabg-F/-R PCR) was gel-isolated, radioactively labeled (³²P-dCTP, MP Biomedicals; Germany) using RediPrime (GE Healthcare; Germany). This was then used for Southern blot hybridization, carried out under conditions of 50° C. in 4×SSPE (1×=0.18 M NaCl, 10 mM PP, 1 mM EDTA, pH 7.4) containing 0.5% (w/v) non-fat milk powder, 1.0% (w/v) sodium.dodecylsulfate (SDS), and 0.5 mg/ml denatured calf thymus DNA (KT-DNA, Sigma; Germany). Following X-ray (Kodak X-Omat; Germany) exposure, the probe was removed from the filter by incubation in 0.4 N NaOH at 45° C. for 30-60 min, followed by brief incubation at 100° C. in 0.1×SSC, 0.5% SDS, 0.2 M Tris-HCl (pH 7.4). For the second hybridization, the HindIII fragment H of D1701-V containing the vegF-E locus was used as described (Cottone et al., 1998. Virus Res. 56, 53-67). Southern blot results confirmed the correct insertion of the G gene into the genome of D1701-VrV.

### Detection of G Gene-Specific RNA.

Vero cells were infected with a MOI of 3-5, and total RNA was isolated at different times after infection (p.i.) using SurePrep Total RNA Extraction Kit (Fisher Scientific; Germany). Additionally, RNA was extracted from cells infected in the presence of cytosine arabinoside (AraC; 0.04 mg/ml, Sigma; Germany) or cycloheximide (CHX, 0.1 mg/ml, Serva; Germany) to test for early expression of the inserted G gene. As a control, RNA was isolated from non-infected cells. The RNAs were separated in a denaturing 1% agarose get containing formaldehyde, and transferred to nylon membrane as described (Kroczek, R. A. & Siebert. E. Anal. Biochem, 184:90-95, 1990). The radioactively-labeled Rabies G PCR (RabG-F/-R) fragment was used as hybridization probe in UltraHyb solution (Ambion; Germany) at 42° C. The results clearly demonstrated immediate early expression of the Rabies G gene, due to its regulation under the control of the early vegF-E promoter of ORFV (Rziha et al. 1999, J. Biotechnol., 73, 235-242)

### Detection of G Protein by Immunofluorescence.

For immunofluorescence, Vero cells (1×10⁵ cells/ml) were infected in 4-chamber slides (BD Falcon; Germany) with a MOI of 0.1 or 3.0. At different times p.i., the cells were washed with medium, and fixed with 3.7% (v/v) methanol-free formaldehyde (Pierce, Thermo Fisher Scientific; Germany) for 15 min at 37° C. After 3 washes with PBS, the cells were permeabilized by treatment with 0.2% (v/v) Triton X-100 for 5 min at 37° C. After PBS washing, the cells were blocked with 5% FCS in PBS for 30-40 min at 37° C. For G protein detection, cells were incubated for 1 hr at 37° C. with the mouse monoclonal antibody G559 (FLI; Tuebingen, Germany) diluted 1:1000 in PBS containing 1% FCS. After 5 washes in PBS, slides were incubated in the dark at 37° C. for 30 min with the secondary anti-mouse Alexa-555 antibody, diluted 1:1000 in PBS (Molecular Probes; Germany).

Detection of cells at late times after infection with ORFV was carried out by the use of ORFV-specific rabbit antiserum PAS2274, provided by Dr. Rudiger Raue, (Pfizer, UK). The serum was diluted 1:100 in PBS with 1% FCS, and secondary antibody, the anti-rabbit Alexa-488, was used at a 1:1000 dilution.

Actin staining was performed with Phalloidin-647, according to the instructions of the manufacturer (Biotium; Germany) followed by staining of the nucleus with 0.04 μg/ml DAPI (4′,6-Diamidin-2′-phenylindoldihydrochlorid; Roche Molecular Biochemicals; Germany), for 20-30 min at RT in the dark. After thorough washing in PBS, the slides were embedded with Mowiol-DABCO, and fluorescence imaging was performed with a Zeiss ApoTome, using Axiovision software.

The results clearly demonstrated strong expression of the Rabies G protein early (4 hr p.i.) as well as late (24 h p.i.) after infection with D1701-V-RabG. Cells infected late could be additionally identified by specific staining with antiserum PAS2274. Moreover, the fluorescence staining proved evidence of surface expression of the G protein. Specificity of staining was tested via the use of non-infected cells.

### Detection of G Protein by Western Blotting

Vero cells (3×10⁵ cells) were simultaneously infected with a MOI of 1.0, and incubated at 37° C. in a 5% CO₂ atmosphere. At different times p.i., the cells plus supernatant were harvested, centrifuged (8,900×g, 10 min, 4° C.), and the cell sediment was washed 3 times with 1.0 ml PBS and resuspended in 0.15 ml PBS containing 1% (v/v) Triton X-100. After 30 min on ice, the lysate was centrifuged for 15 min at 15.000×g, 4° C., and the supernatant saved for SDS-PAGE (Polyacrylamide gel electrophoresis). To this end, 3 parts of lysate were mixed with one part 4× DualColor protein loading buffer (Fermentas; Germany), boiled for 5 min, sonicated, and approx. 10 μg protein was separated by SDS-PAGE using 8% (w/v) ProSieve50 gel with Tris-Tricine-SDS running buffer as recommended (FMC Bioproducts, Biozym; Germany). The Prestained Protein Ladder (Fermentas; Germany) was used as molecular weight markers. After electrophoresis, the proteins were transferred to a PVDF membrane according to the instruction of the manufacturer (Pierce, Thermo Fisher Scientific; Germany). After membrane blocking in 3× Rotiblock (Roth; Germany) for 3 hours at room temperature, a rabbit anti-peptide antiserum specific for the C-terminus of the Rabies virus G protein (kindly provided by Dr. K.-K. Conzelmann, Max-von-Pettenkofer Institute; Munich, Germany) was used 1:10,000, diluted in 1× RotiBlock. After overnight incubation at 4° C., the membrane was thoroughly washed 5 times in TBS-T (Tris-buffered saline with 0.05% v/v Tween-20), and incubated with a peroxidase-coupled anti-rabbit antibody (1:20,000; Jackson-ImmunoRes., Dianova; Germany) for 1 hr at RT. After TBS-T washing, ECL substrate was used as recommended (Immobilon Western, Millipore; Germany). The reacted proteins were detected by the use of chemiluminescence X-ray film (CL-XPosure, Pierce, Thermo Fisher Scientific; Germany).

Expression of the Rabies virus G protein of the expected molecular weight (58-60 kDa) was confirmed at the different times after infection.

### Example 3

### Induction of Specific Immune Response after Immunization of Mice with D1701-V-RabG

### Dose Dependent Induction of Anti-G Serum Antibodies.

The G protein can be regarded as the most important antigen for a protective immune response, and the extent of the induced virus-neutralizing serum antibodies (SNT) can be correlated with protection against Rabies virus challenge infection. According to the OIE (World Organization of Animal Health) and WHO (World Health Organization), the presence of antibody titers exceeding 0.5 to 1.0 IU/ml (International unit) of SNT are regarded as protective. Therefore, the induction of G protein-specific SNT antibodies was determined during day 1 to day 14 following prime immunization of mice with D1701-V-RabG.

C57/BL6 mice of 6-8 weeks of age (n 6 or 7 per group), bred at the FLI (Friedrich-Loeffler-Institute, Federal Research Institute of Animal Health, Institute of Immunology; Tuebingen, Germany), were intramuscularly immunized with 0.1 ml of the indicated PFU (plaque-forming units) of D1701-V-RabG (0.05 ml for the thigh of each hind leg). Individual serum samples were taken daily, and used for determination of SNT in a rapid fluorescence focus inhibition test (RFFIT) as described. (OIE Manual of Diagnostic Tests and Vaccines for Terrestrial Animals. 2007, Part 2, Section 2.2, Chapter 2.2.5 Rabies, which is also found at the following Internet website: www.oie.int/fr/normes/mmanual/A_{—}00044.htm; Cox, J. H. and L. G. Schneider, 1976, J. Clin. Microbial. 3, 96-101.) In brief, serial 5-fold serum dilutions in RPMI medium were prepared, and 0.05 ml of each dilution was mixed (in duplicate) in the wells of a 96-well plate, together with 0.05 ml Rabies virus, strain CVS 11 (lot no. 47, 1.7×10⁵ PFU/ml). After 90 min incubation at 37° C. and 5% CO₂, 0.1 ml of a BHK21 cell suspension (1×10⁶ cells/ml) was added into each well, mixed, and incubated 24 hours at 37° C. in 5% CO₂. After washing the wells of the culture plate with PBS and pre-chilled 80% (v/v) acetone, the cells were fixed in fresh 80% (v/v) acetone for additional 30 min at 4° C. After removal of the acetone and air drying, 0.05 ml of FITC anti-rabies monoclonal globulin (Centocor; USA) was added for 30 min at 37° C. to stain the Rabies virus-infected cells. After washing twice with PBS and once with aqua bidest, the virus infection was monitored by fluorescence microscopy. Serum dilutions that reduced the number of infected cells to 50% were read as positive. The titers of the sera were expressed as IU/ml, and were compared to positive WHO reference serum.

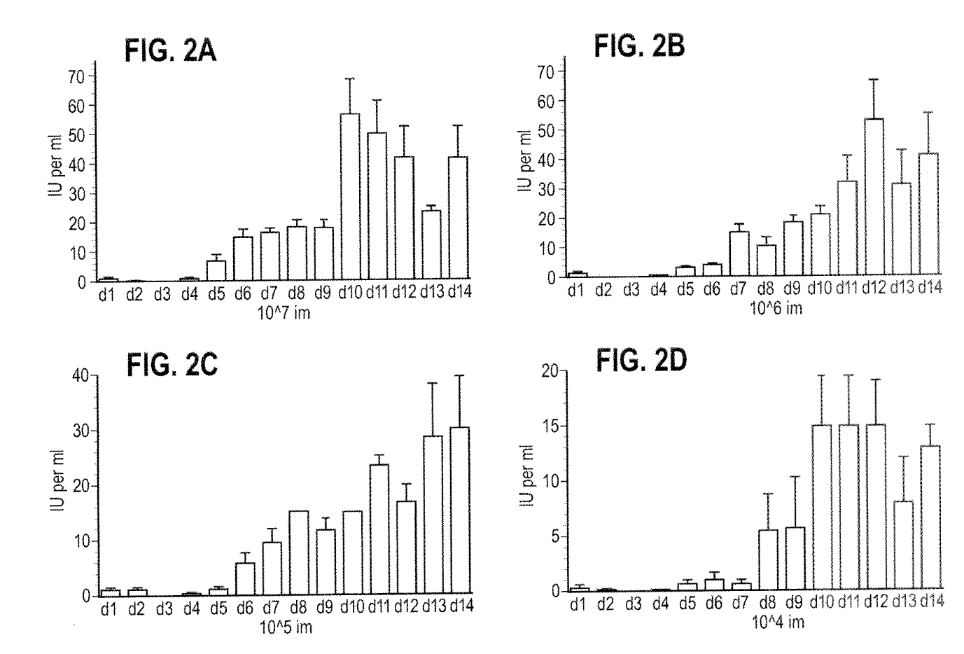

FIG. 2 demonstrates the development of SNT after a single intramuscular immunization of mice with the indicated PFU of D1701-V-RabG recombinant virus. Sera were tested on the indicated days (d) after immunization by FFIT. As can be seen in FIG. 2(D), even low dose (10⁴ PFU) immunization resulted, by day 8, in a mean SNT of 5.5 IU/ml, increasing until day 14 after immunization to approximately 13 IU/ml. Using 10⁶ or 10⁷ PFU for immunization (FIGS. 2A and 2B), by one week later, a mean SNT of approximately 10-20 IU/ml was induced, respectively, which increased up to approximately 50 IU/ml 14 days after priming. Using 10⁷ PFU of D1701-V-RabG, by day 4 after immunization, protective serum antibody titers (0.6-3.0 IU/ml) were detected, which was not the case using the tested lower immunization doses (FIG. 2).

Another mouse experiment (data not shown) demonstrated that a booster immunization using either 10⁶ or 10⁷ PFU of the recombinant virus led to only a marginal increase in the SNT 14 days after prime immunization (with 10⁶ or 10⁷ PFU).

Collectively, these results demonstrate the successful induction of protective SNT antibody titers during the first week after immunization of mice with various doses of D1701-V-RabG.

### Example 4

### Protective Immune Response in Mice Mediated by D1701-V-RabG

An evaluation of the protective capacity of D1701-V-RabG was performed by challenge infection of different immunized mice (C57/BL6) according to the recommendations of the DIE (Manual of Diagnostic Tests and Vaccines for Terrestrial Animals, 2007. Part 2, Section 2.2, Chapter 2.2.5 Rabies. Also found at www.oie.int/fr/normes/mmanual/A_{—}00044.htm.). At an age of 6-8 weeks, mice (n=11 or 12 per group) were immunized with the indicated PFU of D1701-V-RabG as described above. Non-immunized control mice (n=15) were injected with PBS. Three weeks after immunization, all animals were intracranially challenged with virulent rabies virus strain CVS (0.03 ml containing 4.8×10⁵ PFU). The animals were observed daily until 21 days after challenge infection. Moribund animals suffering from severe neuronal symptoms were euthanized.

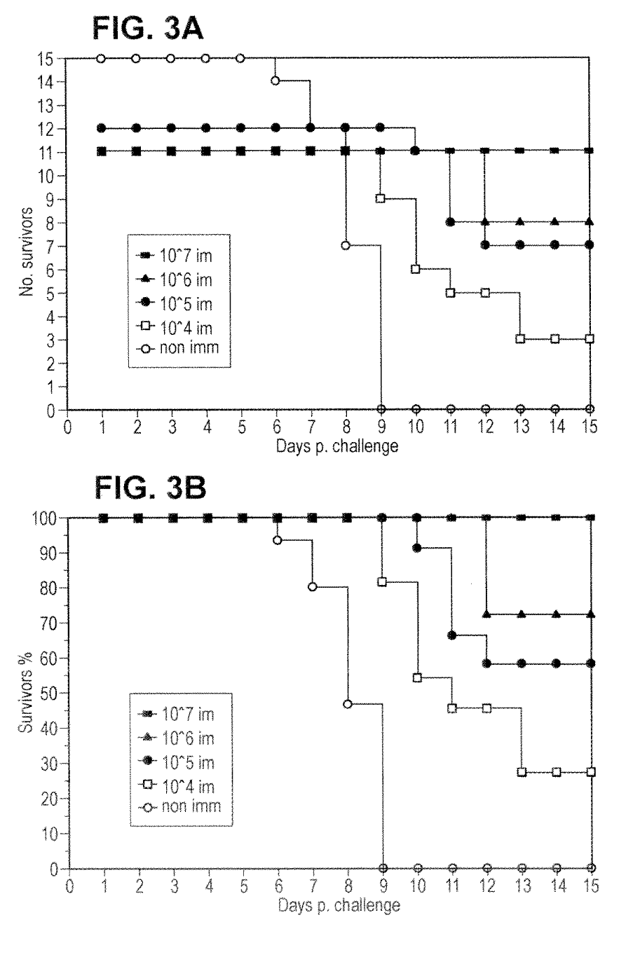

As shown in FIG. 3, a single intramuscular immunization with 10⁷ PFU of D1701-V-RabG completely protected the mice (11/11) against a high dose of intracranially applied challenge virus. One immunized animal from that group died on day 12, but this was not due to the challenge Rabies virus infection. Therefore, that animal was excluded from the data analysis. Application of one dose containing a 10-fold lesser amount (10⁶ PFU) of D1701-V-RabG still achieved 73% protection (8/11 survivors). Further decrease of the immunization dose reduced the protective rate to 58% (10⁵ PFU; 7/12), or to 27% (10⁴ PFU; 3/11), whereas all control immunized mice (n=15) died during day 6 and 9 after challenge infection (FIG. 3).

### Example 5

### Role of T-Cells for Protective Immunity

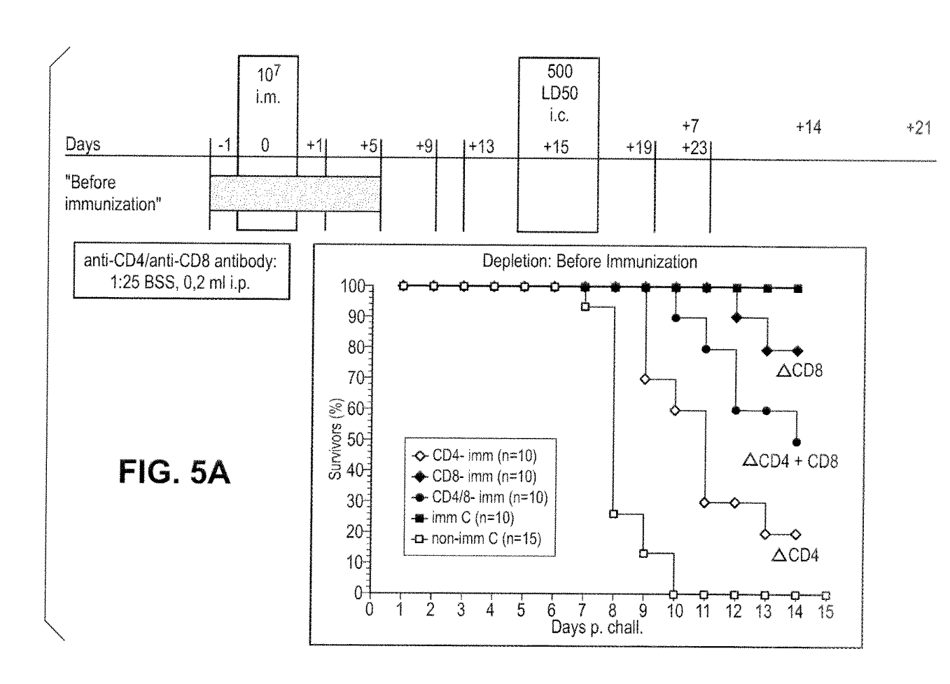

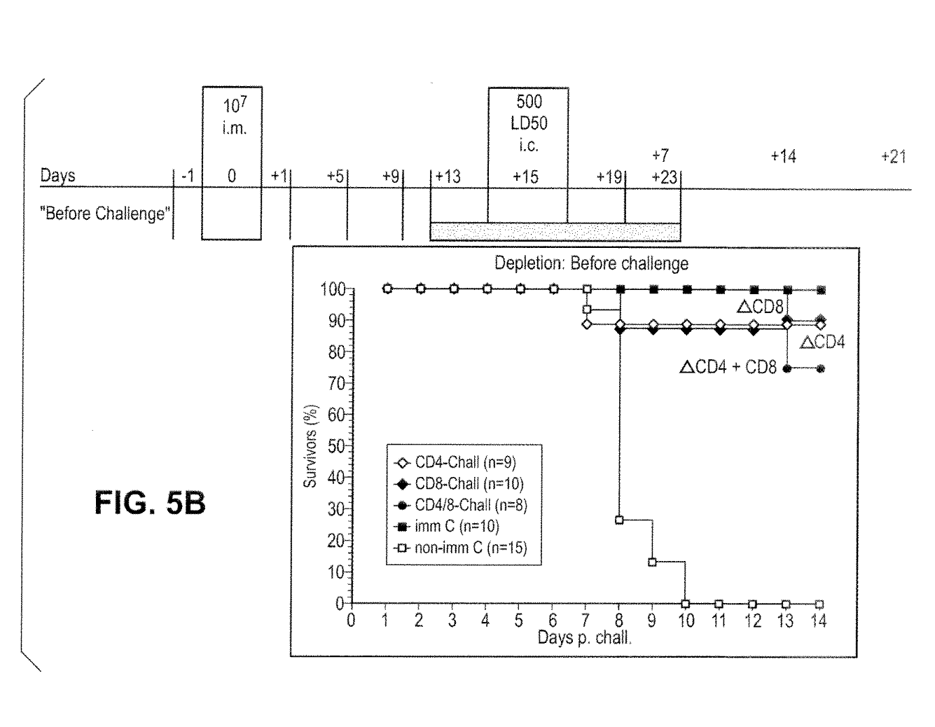

The following experiments investigated the contribution of T cells (CD4-, CD8-, or CD4/8-positive cells) for the induction of protective immunity by the recombinant D1701-V-RabG in mice. As indicated in FIG. 5A, just prior to and following the Day-0 immunization with 10⁷ PFU of D1701-V-RabG (i.e., on Days −1, 0, +1 and +5), antisera specific for CD4- or CD8-T-cells were administered intraperitoneally to groups of mice (number (n) of animals shown in the Figure) to deplete each T-cell population in vivo.

Thereafter, on Day 15, all animals were challenged intracerebrally with a dose of 500 LD₅₀ of virulent RV strain CVS.

As shown in FIG. 5A, animals depleted for CD4-positive T-cells generally were not protected, as seen by the similar response to the non-immunized control animals.

As seen in FIG. 5B, groups of animals were immunized on Day 0 with D1701-V-RabG, and then in vivo depleted of CD4- and/or CD8-T-cells just prior to and following the Day-15 challenge infection with the virulent RV strain CVS, i.e., on Days 13 (2 days before challenge infection), 15, 19, and 23. The results demonstrate that after successful priming of the anti-Rabies virus immune response by D1701-V-RabG, the depletion of T-cells 14 days later did not adversely affect protection, in that 75-90 percent of animals that were in vivo depleted of T-cell populations survived the challenge.

Conclusively, the results show that CD4-positive T-cells (most probably T-helper cells necessary for anti-G antibody production) are the major determinant of the protective immunity induced by the recombinant D1701-V-RabG.

### Example 6

### Post-Exposure Vaccination

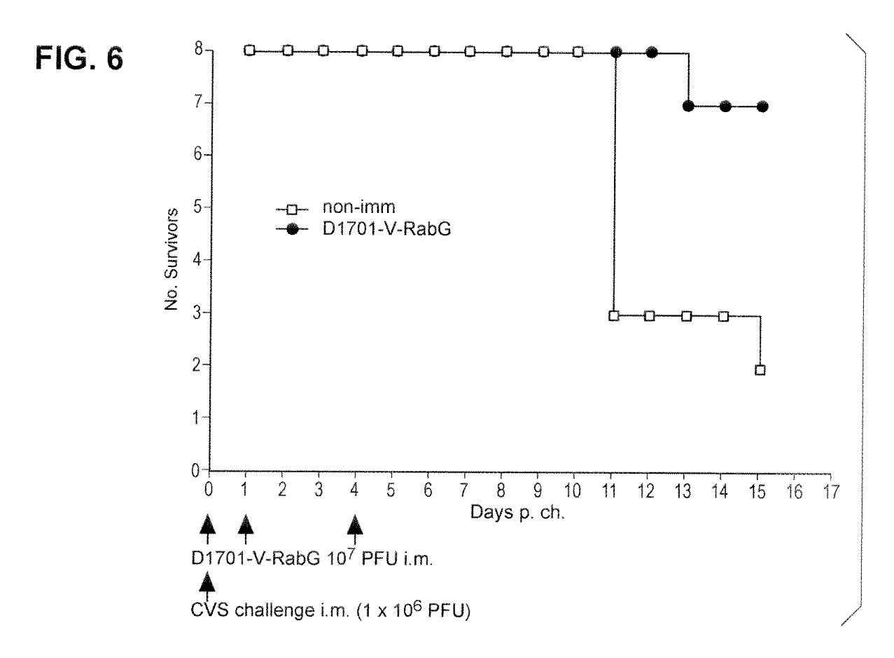

The efficacy of the recombinant D1701-V-RabG for therapeutic vaccination was tested in mice. To this end, groups of animals were vaccinated intramuscularly (i.m.) with 10⁷ PFU of D1701-V-RabG on Days 0, 1, and 4. The mice were challenged with 1×10⁶ PFU of virulent RV strain CVS on Day 0. As shown in FIG. 6, all mice in the immunized group except one were protected against rabies.

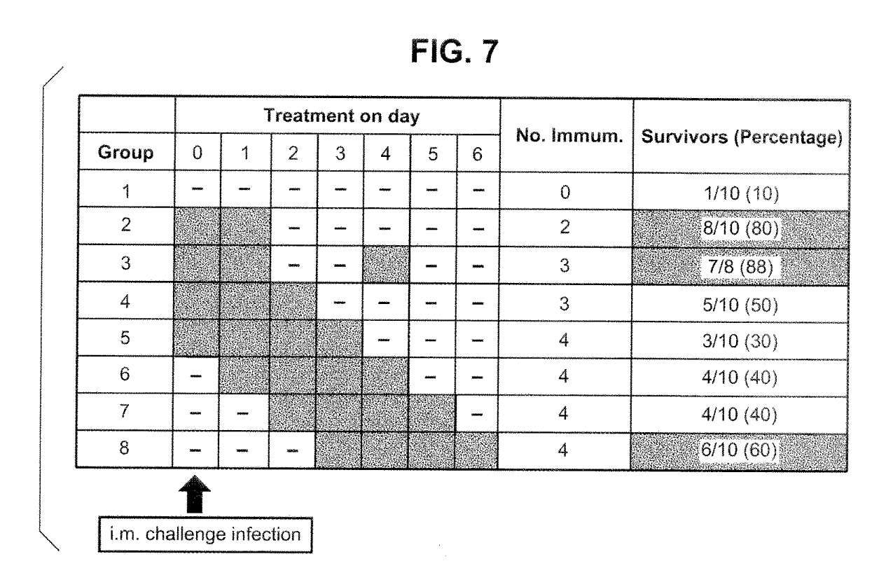

Additional experiments were performed in mice to test various post-exposure immunization regimens to the virulent RV strain CVS. In FIG. 7, gray squares indicate the days on which mice were vaccinated with D1701-V-RabG. Results (survivors) are summarized in the Figure.

The results demonstrate the capability of D1701-V-RabG for therapeutic vaccination of mice. As seen in FIG. 7, a dual vaccination, given on the day of challenge and the following day, mediated 80% protection, and at least 60% of animals were protected by 4 daily immunizations beginning 3 days after the peripheral RV challenge infection.

### Example 7

### Immune Response Induced by Different Routes of Immunization

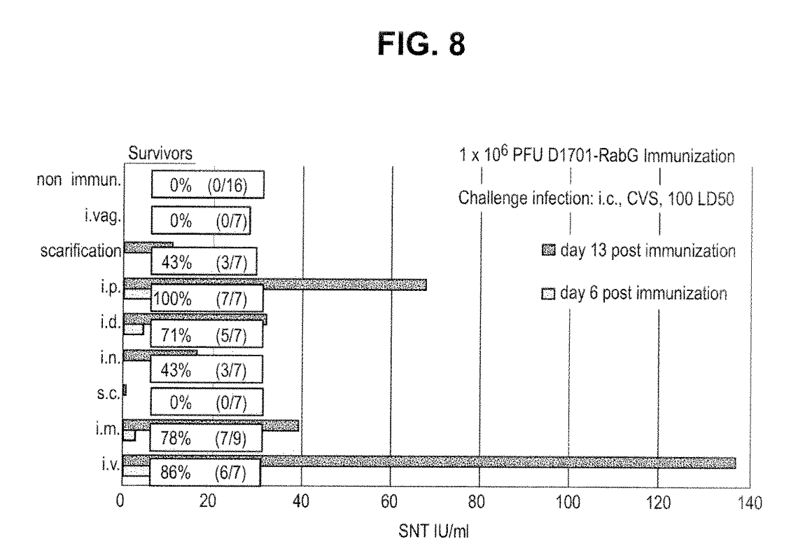

Mice were immunized with 1×10⁶ PFU of D1701-V-RabG by the following of application: intravaginally (i.vag.), by scarification, intraperitoneally (i.p.), intradermally (i.d.), intransally (i.n.), subcutaneously (s.c.), intramuscularly (i.m.), and intraveneously (i.v.). The induced serum antibody response (determined as serum neutralization antibodies, or SNT) 6 days (gray bars) and 13 days (black bars) after immunization is depicted in FIG. 8. Fourteen days after immunization the animals were intracerebrally infected with 100 LD₅₀ of virulent RV strain CVS, and the percent survivors was calculated.

The results show that the highest SNT titers (given as IU per ml) were induced by i.v., i.p., and i.m. vaccination, which also resulted in the best protection rates of 86%, 100%, and 78%, respectively.

What is claimed:

1. A recombinant parapoxvirus comprising a parapoxvirus and heterologous DNA derived from a rabies virus.

2. The recombinant parapoxvirus of claim 1, wherein the parapoxvirus is a Parapoxvirus ovis virus (ORFV).

3. The recombinant parapoxvirus of claim 2, wherein the parapoxvirus is Parapoxvirus ovis strain D1701.

4. The recombinant parapoxvirus of claim 1, wherein the heterologous DNA comprises the gene encoding the G protein of the rabies virus, or fragments thereof.

5. The recombinant parapoxvirus of claim 1, wherein the heterologous DNA comprises SEQ ID NO: 4, or a sequence having at least about 98% identity to SEQ ID NO: 4.

6. The recombinant parapoxvirus of claim 1, wherein the heterologous DNA is inserted within the HindIII fragment H/H of Parapoxvirus ovis strain D1701.

7. The recombinant parapoxvirus of claim 6, wherein the heterologous DNA is inserted within the VEGF coding sequence or adjacent non-coding sequences within the HindIII fragment HUH of Parapoxvirus ovis strain D1701.

8. The parapoxvirus of claim 1, wherein the parapoxvirus is Parapoxvirus ovis D1701-V-RabG.

9. A method of preparing the recombinant parapoxvirus of claim 1 comprising inserting heterologous DNA into the genome of the parapoxvirus.

10. The method of claim 9, wherein the parapoxvirus is Parapoxvirus ovis.

11. The method of claim 10, wherein the parapoxvirus is Parapoxvirus ovis strain D 1701.

12. The method of claim 9, wherein the heterologous DNA comprises the gene encoding the G protein of the rabies virus, or fragments thereof.

13. The method of claim 9, wherein the heterologous DNA comprises SEQ ID NO: 4, Or a sequence having at least about 98% identity to SEQ ID NO: 4.

14. The method of claim 9, wherein the recombinant parapoxvirus is Parapoxvirus ovis D1701-V-RabG.

15. An immunogenic composition comprising the recombinant parapoxvirus of any of claims 1 to 8 and a carrier.

16. A method of preparing the immunogenic composition of claim 15, comprising combining the recombinant parapoxvirus with a carrier.

17. A method of inducing an immune response against rabies virus in an animal comprising administering to said animal an immunologically effective amount of an immunogenic composition of claim 15.

18. The method of claim 17, wherein the immune response is the induction of anti-G protein serum antibodies.

19. The method of claim 17, wherein an anti-G protein-specific protective immune response is induced.

20. The method of claim 19, wherein the induction results in antibody titers exceeding 0.5 International Units per ml.

21. Use of the recombinant parapoxvirus of any one of claims 1 to 8 in the preparation of a medicament for inducing an immune response against rabies virus in an animal.

22. A use of the recombinant parapoxvirus of any one of claims 1 to 8 in an assay for the differentiation of infected from vaccinated animals.


SEQUENCE LISTING (9 sequences)

SEQ ID NO: 1 - DNA, 30 bp, Artificial Sequence
  primer
gcgcgctgcgggtgcgctaccaattcgcgc

SEQ ID NO: 2 - DNA, 32 bp, Artificial Sequence
  primer
gcatcccgttaccaccggagaccgacgctccc

SEQ ID NO: 3 - DNA, 86 bp, Parapoxvirus ovis
  misc_recomb (1)..(86): D1701 viral sequences plus multiple cloning site facilitating integration of recombinant gene into viral genome
aattataacgcccatcccgggataagcttgatatctgcaggatccaggtaccgaattcatcgtcgacttcgagagcttagaatgcg

SEQ ID NO: 4 - DNA, 1588 bp, Neurotropic lyssavirus
  gene (1)..(1588): Rabies G gene - Full length coding region plus 7 nt on the 5′-end and 6 nt on the 3′-end
ggatccgatggttcctcaggctctcctgtttgtaccccttctggtttttccattgtgttttgggaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatctcagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccactggcttcgaactgtaaaaaccaccaaggagtctctcgttatcatatctccaagtgtggcagatttggacccatatgacagatcccttcactcgagggtcttccctggcgggaattgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccggtcagttggtgaatttgcacgactttcgctcagacgaaattgagcaccttgttgtagaggagttggtcaagaagagagaggagtgtctggatgcactagagtccatcatgaccaccaagtcagtgagtttcagacgtctcagtcatttaagaaaacttgtccctgggtttggaaaagcatataccatattcaacaagaccttgatggaagccgatgctcactacaagtcagtcagaacttggaatgagatcatcccttcaaaagggtgtttaagagttggggggaggtgtcatcctcatgtaaacggggtatttttcaatggtataatattaggacctgacggcaatgtcttaatcccagagatgcaatcatccctcctccagcaacatatggagttgttggtatcctcggttatcccccttatgcaccccctggcagacccgtctaccgttttcaagaacggtgacgaggctgaggattttgttgaagttcaccttcccgatgtgcacgaacggatctcaggagttgacttgggtctcccgaactgggggaagtatgtattactgagtgcaggggccctgactgccttgatgttgataattttcctgatgacatgctggagaagagtcaatcgatcggaacctacacaacacaatctcagagggacagggagggaggtgtcagtcactccccaaagcgggaagatcatatcttcatgggaatcatacaagagcgggggtgagaccggactgtgagaattc

SEQ ID NO: 5 - DNA, 7692 bp, Artificial Sequence
  plasmid
tatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggcccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcccagctggcttatcgaaattaatacgactcactatagggagaccggaatttgcaggatcgcgctgctgtacctgaagatatacgagctcccgggctgcacgaacttcctgcacgtggacctgaagcccgacaacgtgctcatcttcgacagcgcgcgcgcgctcagcgtgactgcggccggtgcgacttttcgcttcgaagagcccgtgcgcgcggcgctgaacgacttcgacttcgcgcgcgtggccaccatcgagaaccgcaagatcgcgggcagcgtccgcgtgccgcagaactggtactacgacttccacttcttcgcgcacacgctgctgcgcgcgtacccgcacatcgccgcggaggacccgggcttccacgcgctgctctcggagctcacggtctcgtgctcgcgcgggacctgcgaccgcttccggctgcgcgtgtcctcgccgcaccccatcgagcacctcgcgcggctggtgcgccgcgacgtcttctcccgctggataaatgccgccgcggacgcccccgacgccgcactctcctgagcccacgcccgcggcgccgggctcgctgtacgacgtcttcctcgcgcgcttcctgcgccagctggccgcgcgcgcggcgccggcctcggccgcctgcgccgtgcgcgtgggtgcggtgcgcggccgcctgcggaactgcgagctggtggtgctgaaccgctgccacgcggacgctgccggcgcgctcgcgctggcctccgcggcgctggcggaaacgctggcggagctgccgcgcgcggacaggctcgccgtcgcgcgcgagctgggcgtggacccagagcacccggagctgacgccggaccccgcctgcgcgggcgagagcgcgcttgcgcagaacatcgacatccagacgctggacctgggcgactgcggcgaccccaaaggccgccgactgcgcgtggcgctggtgaacagcggccacgcggccgcaaactgcgcgctcgcgcgcgtagcgaccgcgctgacgcgccgcgtgcccgcaagccggcacggcctcgcggagggcggcacgccgccgtggacgctgctgctggcggtggccgcggtgacggtgctcagcgtggtggcggtttcgctgctgcggcgcgcgctgcgggtgcgctaccaattcgcgcggccggccgcgctgcgcgcgtagccgcgcaaaatgtaaattataacgcccatcccgggataagcttgatatctgcaggatccgatggttcctcaggctctcctgtttgtaccccttctggtttttccattgtgttttgggaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatctcagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccactggcttcgaactgtaaaaaccaccaaggagtctctcgttatcatatctccaagtgtggcagatttggacccatatgacagatcccttcactcgagggtcttccctggcgggaattgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccggtcagttggtgaatttgcacgactttcgctcagacgaaattgagcaccttgttgtagaggagttggtcaagaagagagaggagtgtctggatgcactagagtccatcatgaccaccaagtcagtgagtttcagacgtctcagtcatttaagaaaacttgtccctgggtttggaaaagcatataccatattcaacaagaccttgatggaagccgatgctcactacaagtcagtcagaacttggaatgagatcatcccttcaaaagggtgtttaagagttggggggaggtgtcatcctcatgtaaacggggtatttttcaatggtataatattaggacctgacggcaatgtcttaatcccagagatgcaatcatccctcctccagcaacatatggagttgttggtatcctcggttatcccccttatgcaccccctggcagacccgtctaccgttttcaagaacggtgacgaggctgaggattttgttgaagttcaccttcccgatgtgcacgaacggatctcaggagttgacttgggtctcccgaactgggggaagtatgtattactgagtgcaggggccctgactgccttgatgttgataattttcctgatgacatgctggagaagagtcaatcgatcggaacctacacaacacaatctcagagggacagggagggaggtgtcagtcactccccaaagcgggaagatcatatcttcatgggaatcatacaagagcgggggtgagaccggactgtgagaattcatcgtcgacttcgagagcttagaatgcgtccccacggaagaggcaaacgtaacgatgcaactcatgggagcgtcggtctccggtggtaacgggatgcaacatctgagcttcgtagagcataagaaatgcgattgtaaaccaccactcacgaccacgccaccgacgaccacaaggccgcccagaagacgccgctagaactttttatggaccgcatatccaaacgatgatgcgatcaggtcatgcggaaggaggctccacggagcaaagtgaaaaaggaccgcctagagtcgagacccctccctcccgcctcgggcaaacccacagccgccgcaaacaccacacccgccgacctaccatgcacccctcgccgcgccggctgctcggcgcgctcgcgctgctggcgctgggcttcgctcggcgcgctcttcgccccgcggcgccgctcgtgccggccgccttcctggaggtggggcacgtgcgcgcgaacccgtccgcctcggtgacctgcctcacggtgggcggcgacgggcggcacatggcggcggtcgcgcacggcggcgggacgctctcgccggtgtacccgctggccgccggcatgcacgcgaccttctcctccgcgcgcaagggcgcgctgctgctgaacgtcgcgaccgtgactgtgtacgacgtgcgcgcgctcgcccccgagttcgagctcgtctgcatcgcggtggtcggcggctacaactcggccgcggccgccacgcggcccgcggccgagtggcaccgccagctggagctgcgccgctcggagctgtgacccctccctccccggtctccctctgtctttgtaatcggccttagagattagacatcatcctccacgcctctttgtccgccgcccttcttcgcggacggatgaaccaattaattaattatttttgtcgctcgcccgctcactccggcaagggaacgagtgacgttaactctctcaccctcacgcacaagaacaagaaccgctcactcaccgggcaagggaacacggttaaggtcaactcactcgcgagaacaagttgaccctcactctagagaacgaggaacgggcaacaagcaaccgtcaactcacttaccacgagaacaagttgaccgccactcaaagggaacagagaacagtaaccgttctcgctcgctcggaacaatagaacaagttaacgtcaactcgctcgctcggtgtaagagaacaacagaacaagcaactgttgaccactcaacccccggagaagagaacaagagagcagtcaactcacccactcagtcttggatgagaggaggacgagttaacgagtactcgcacgcagagtgagagagtgaggacataataatagttaacgagttaatactcactcgctcactcagagtgagagagaaccagtgagcgagttaaccgcgcacacgagcgagagaacagtgaactgctcgcgcgctcgctcggtagcagtcggcctttcttaaaacggttcgtaaaacttttcccgagacagttcaccctccaaaacttttaaaactaaactcggaggtggcctgccctccactctccgtaaaacttttgtaaaactgtcggaggtcggtcgacttcgcaactcgtccgcgaaaacttttcgtgggcagtgtctgcctctctcaggctcctcgcatcactttcgcggagcctcgaggtaggtcacctctctccaaacttttgtaaaaactttttcgcggagcctctggaggccgtcctccctccaaaacttttcgtaaaatctcttcggaggccgtcctccctccaaaacttttcgtaaaatctttgggaggtcgacctccctcaaaactttttataaagtagcttgtattc

SEQ ID NO: 6 - DNA, 23 bp, Artificial Sequence
  primer
ggagtctctcgttatcatatctc

SEQ ID NO: 7 - DNA, 23 bp, Artificial Sequence
  primer
ctaaacaaggtgctcaatttcgt

SEQ ID NO: 8 - DNA, 23 bp, Artificial Sequence
  primer
cgatactgtcgtcgtcccctcaa

SEQ ID NO: 9 - DNA, 22 bp, Artificial Sequence
  primer
caactcgccgcacatctgaact